(19) United States
(12) Patent Application Publication
(10) Pub. No.: US 2024/0398219 A1
(43) Pub. Date: Dec. 5, 2024

(54) ENDOSCOPIC INSTRUMENT

(71) Applicant: Richard Wolf GmbH, Knittlingen (DE)

(72) Inventors: Bernd PFITZENMEIER, Bretten (DE); Alexander FRANK, Oberderdingen (DE)

(21) Appl. No.: 18/262,765

(22) PCT Filed: Feb. 3, 2022
(86) PCT No.: PCT/EP2022/052555

§ 371 Date: Jul. 25, 2023

(30) Foreign Application Priority Data
Feb. 5, 2021 (EP) 21155375.5

(51) Int. Cl. A61B 1/05; A61B 1/00; H01L 27/146

(52) U.S. Cl. CPC A61B 1/051; A61B 1/00103; A61B 1/0011; H01L 27/14625

(57) ABSTRACT

An endoscopic instrument (48) includes a tubular shaft (63) with an objective lens (1, 45), arranged at a distal tip (49), with an arrangement (3) of connected lens elements (5, 7, 9, 11, 13) having optical properties and following each other along an optical axis (15). The arrangement has a polygonal and at least hexagonal cross-section that is perpendicular to the optical axis. The lens is inserted interlocking, friction-locking and/or bonded in an imaging channel (51) of the distal tip. The imaging channel includes a first distal imaging channel section (51a) into which the objective lens is inserted, and a second imaging channel section (51b), with a greater inner diameter, arranged proximally of the first imaging channel section. An image sensor unit (17) bond connected to the objective is arranged in the second imaging channel section. The image sensor unit has greater lateral dimensions than the objective lens.

### CROSS REFERENCE TO RELATED APPLICATIONS

This application is a United States National Phase Application of International Application PCT/EP2022/052555, filed Feb. 3, 2022, and claims the benefit of priority under 35 U.S.C. § 119 of European Application 21155375.5, filed Feb. 5, 2021, the entire contents of which are incorporated herein by reference.

### TECHNICAL FIELD

The disclosure relates to an endoscopic instrument as well as a method of manufacturing endoscopic instruments.

### BACKGROUND

For minimising the radial extent of an imaging channel in the shaft of the instrument, objective lenses for image sensors of an endoscopic instrument usually have a very thin diameter of 1 mm or less. The use of camera modules with an image sensor unit and an objective lens arranged thereon for integration into such an instrument is known. A generally known method of manufacturing miniaturised objective lenses is based on the principle of so-called Wafer Level Optics (WLO). Here, several substrate layers with predetermined optical properties are combined with each other and form a wafer package from which individual objective lenses are cut out. The separating process is often orientated to the size and shape of the image sensor. This is usually quadratic so that the objective lens acquires a corresponding cross-section. If such a known WLO lens is now to be used in an endoscopic instrument in an imaging channel with less than 1 mm diameter with a sufficiently large optically effective area, it must initially be over-dimensioned and then circumferentially ground off or rounded off to the size of the imaging channel. However, because of the small dimensions of the lens, grinding off or rounding off is laborious as a grinding procedure only results in a precision component in the case of exact centring. This leads to significant additional costs and consequently to a correspondingly higher unit price.

### SUMMARY

An aim of the invention is to provide an endoscopic instrument with an objective lens, wherein the objective lens is particularly compact in the radial direction, but nevertheless has a largest possible optically effective surface and at the same time is cost-effective to manufacture.

According to a first aspect of the present disclosure, an endoscopic instrument with a tubular shaft is provided, at the distal end of which an objective lens is arranged, wherein the objective lens comprises an arrangement of objective lens elements separated out of a multilayer wafer package and connected to each other, which each have predetermined optical properties and follow each other along an optical axis. In accordance with the invention, it is envisaged that the objective lens has a polygonal and at least hexagonal cross-section perpendicular to the image axis. The distal tip has an imaging channel, wherein the objective lens is inserted into the imaging channel in an interlocking, friction-locking and/or materially bonded manner. The imaging channel comprises a first distal imaging channel section, into which the objective lens is inserted, and, arranged proximally of the first imaging channel section, a second imaging channel section which has a larger internal diameter than that of the first imaging channel section, wherein an imaging sensor unit materially bonded to the objective lens is arranged in the second imaging channel section, wherein the image sensor unit has larger lateral dimensions than the objective lens.

The objective lens is thus also produced by means of the Wafer Level Optics (WLO) method of construction. The multilayer wafer package comprises several layers of an optical material. The required layers can be produced with different methods and can be designed differently. It not absolutely necessary for all the layers to be made of the same material. As an example, one layer of the wafer package that provides lenses could be made of a UV-hardening polymer. In turn, a glass substrate could be provided as a carrier or basis of this layer. Depending on the task of the respective layer, other materials can also be selected. In doing so, it should only be ensured that the desired optical properties can be brought about and that the resulting wafer package can be cut in the desired way.

The individual layers are stacked along the subsequent image axis and connected to each other. The connection can be brought about, for example, by way of an adhesive, an anodic connection or other materially bonded connection method. The wafer package allows a large number of objective lens to be produced at the same time in that arrangements of objective lens elements are produced layer-wise and are then cut out of the wafer package.

According to the invention it is envisaged that the arrangement has a polygonal and at least hexagonal cross-section. The WLO method of construction of the objective lenses known in the prior art can lead to rectangular and, more particularly, quadratic cross-sections and result in very large dimensions in relation to the active optical surface. However, through the at least hexagonal cross-section, a contour can be created which comes considerably closer to a circle than a square does. Consequently, post-processing work to adjust the cross-section is not necessary. In addition to the imaging channel, no separate edging of the objective lens is required, as through the contour that approximates a circle, direct fitting into the imaging channel is possible. Compared with quadratic WLO objective lenses, the design as a polygon only requires additional work when cutting the wafer. However, through advantageous arrangement of the objective lens elements on the corresponding layers of the wafer package, the offcuts can be kept relatively low. When using a six-sided and, in particular, hexagonal cross-section, it could be sufficient to cut the wafer package with sequences of parallel cuts in three directions. The optically active surface can be considerably increased in comparison with a quadratic objective lens fitted into an imaging channel.

In a particularly preferably embodiment, the cross-section is equilaterally polygonal and preferably hexagonal or octagonal. Through the equilateral arrangement, the work on separating out the wafer package is further reduced. Especially in the case of the hexagonal shape, the offcuts can be considerably reduced if the objective lens elements in the individual layers of the wafer package are offset with regard to each other, for example in columns, by half the height of the respective cross-section.

Furthermore, it is advantageous if the objective lens elements comprise a first end plate and a second end plate, which delimit the arrangement at face ends that are opposite each other. The end plates could, for example, be made of glass, quartz glass or a polymer, such as a UV-hardening polymer, and delimit the objective lens at the face end. A lens or another light-bundling objective lens element can be axially encompassed and protected by the two end plates. Through the end plates, flat end surfaces with a desired abrasion resistance for the distal end and a suitable optical in-coupling surface for an image sensor at the proximal end are provided at both sides.

Preferably the objective lens elements comprise at least one lens. The lens, as an essential component for optical imaging, can be adapted to the respective form of embodiment of the endoscopic instrument. It is conceivable for the lens to be made of a plastic, such as a UV-hardening polymer. For manufacturing, a lithography method or suchlike could be used on a glass substrate as the support.

It is advantageous if the lens has as an aspherically-shaped surface which is surrounded by a flat edge, wherein a spacer is arranged on the edge and extends outwards along the optical axis over the spherical surface. The spacer protects the lens and can separate it axially from the adjoining objective lens elements. It is conceivable for an adapted fluid to be arranged or enclosed between the lens and the spacer in order to avoid condensation. Through the spacer, the lens is protected in the radial direction, even without circumferential edging.

The lens could be arranged on a flat and continuous surface of a glass substrate. The glass substrate could be a base for constructing a lens body from a polymer. Through mounting the lens directly on the glass substrate, very good optical coupling of the lens and the glass substrate is achieved, which can result in improved connection with adjoining objective lens elements.

Furthermore, the objective lens elements can have a diaphragm that is arranged in the optical axis in front of the lens. Through the diaphragm, the cross-section of bundles of rays that pass through the diaphragm and hit the image sensor are restricted. In this way, among other things, the depth of field of the image can be determined. For a particularly compact embodiment, the diaphragm could be configured as a coating of one of the objective lens elements, wherein the coating is locally interrupted. The coating material can be a light-impermeable material, such as a metal, a metal oxide or a polymer containing pigments and polymers. The diaphragm can, for example, be applied by physical vapour deposition (PVD). The diaphragm function is brought about through the dimensioning of an opening centred in the optical axis. The diaphragm could preferably be arranged between the first end plate and the glass substrate. Preferably, the diaphragm can have a front side and a rear side in relation to the optical axis, wherein the front side and/or the rear side have a light reflectance value of less than 10%. This is particularly advantageous for the image quality, as reflections on the front and rear side of the diaphragm can lead to undesirable image effects. The light reflectance value is defined here as the portion of reflected light power of light power hitting the diaphragm.

Moreover, apart from the imaging channel, the objective lens can be free of its own edging. The objective lens elements are connected to each other and are initially provided without their own separate circumferential edging. In the radial direction the objective lens can take on a maximum possible size and can be directly inserted into the imaging channel of the distal shaft tip. Through the polygonal shape of the cross-section, interlocking, friction-locking and/or materially bonded receiving and fitting into an imaging channel of the endoscopic instrument can take place. The effective optical area of an image sensor is increased.

According to the invention, an image sensor is arranged on the objective lens and configured to record an optical image provided by the objective lens and provide it in the form of electrical signals. Whereas in a possible other variant, the objective lens could be coupled by means of an image conductor, i.e. an ordered bundle of light guide fibres for optional image transmission, to a proximally arranged image sensor, an image sensor could preferably be optically connected directly to the objective lens. An electrical line now only has to be arranged between the image sensor and a device that receives image information. Preferably the image sensor is only connected to a proximal side of the objective lens in a materially bonded manner after the objective lens is cut out of the wafer level package. In this way, if applicable, the lateral dimensions of the objective lens can be made smaller than the lateral dimensions of the image sensor.

In an advantageous form of embodiment, it is envisaged that at least one distal section of the shaft, which comprises the objective lens, is designed as a disposable article. Through the particularly cost-effective manufacturing of the objective lens, costly cleaning and reutilisation of the distal section can be omitted. It could be connected by way of a suitable connection with a proximal section of the endoscopic instrument and detached after use in order to dispose of it.

The shaft of the endoscopic instrument comprises an imaging channel and preferably at least two working channels, wherein the objective lens is inserted into the imaging channel in an interlocking, friction-locking and/or materially bonded manner. Because of the at least six-sided shape of the cross-section, this is closer to a circular shape of the imaging channel than a rectangular and in particular, quadratic cross-section. The six or more corners can radially easily nestle into the wall of the imaging channel and centre the objective lens without causing larger material stresses in the imaging channel and in the objective lens. Mechanically, friction-locking insertion can be easily achieved.

Particularly preferably, the objective lens is adhered to a distal opening of the imaging channel in a fluid-tight manner and thereby outwardly seals off the imaging channel. As a result, no further measures are necessary to seal off the imaging channel for use. In combination with the cost-effective and very precisely fitting production of the objective lens, overall only very modest costs are incurred for the imaging channel of an endoscopic instrument, which also supports the design as a disposable article.

According to the invention, the imaging channel comprises a first distal imaging channel section into which the objective lens is inserted, and, arranged proximally of the first imaging channel section, a second imaging channel section which has a larger diameter than the first imaging channel section, wherein an image sensor unit materially bonded with the objective lens is arranged in the second imaging channel section. The image sensor unit having laterally larger dimensions, can thereby be taken up by the second imaging channel section while the objective lens fits precisely into the imaging channel section.

The invention also relates to a method of producing endoscopic instruments. The method is characterised by the steps of providing several wafer layers of an optical material, stacking the layer to form a wafer package, and separating out objective lenses from the wafer package by cutting the wafer package with at least three groups of parallel separating cuts, the incision directions of which are determined by a polygonal and at least hexagonal cross-section of all objective lenses. In addition, there follows a step of materially bonding each of objective lenses with an image sensor unit, wherein the image sensor unit has greater lateral dimensions than the respective objective lens. Finally there follows a step of inserting the objective lenses materially bonded with the image sensor unit into an imaging channel of a distal tip of the endoscopic instruments to be manufactured, wherein the objective lens is distally inserted into the imaging channel in an interlocking, friction-locking and/or materially bonded manner, wherein the imaging channel comprises a first distal imaging channel section into which the objective lens is inserted, and, arranged proximally of the first imaging channel section, a second imaging channel section which for receiving the image sensor unit has a greater inner diameter than the first imaging channel section.

In an advantageous form of embodiment, when separating out the objective lenses, at least three groups of parallel separating cuts are made, wherein a first incision direction and a second incision direction as well as the second incision direction and a third incision direction each enclose an angle of 60°. In this way, hexagonal cross-sections can be produced.

The invention will be described in more detail below by way examples of embodiment shown in the drawings. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 and FIG. 2 are perspective views of an objective lens with an image sensor unit arranged thereon;

FIG. 3 is a perspective view of an objective lens without the image sensor unit;

FIG. 4 is an exploded view of the objective lens;

FIG. 5 is a schematic view of the effective image sensor surface on the cross-section of the objective lens;

FIG. 6 is an exploded view of the objective lens;

FIG. 7 and FIG. 8 are schematic views of separating the objective lenses out of a wafer package with a hexagonal cross-section (FIG. 7) and octagonal cross-section (FIG. 8);

FIG. 9a, FIG. 9b, FIG. 9c and FIG. 9d are various views of a distal end of an endoscopic instrument with an objective lens arranged thereon; and

FIG. 10a, FIG. 10b and FIG. 10c are various views of a distal end of an endoscopic instrument with an objective lens arranged on the distal tip.

### DESCRIPTION OF PREFERRED EMBODIMENTS

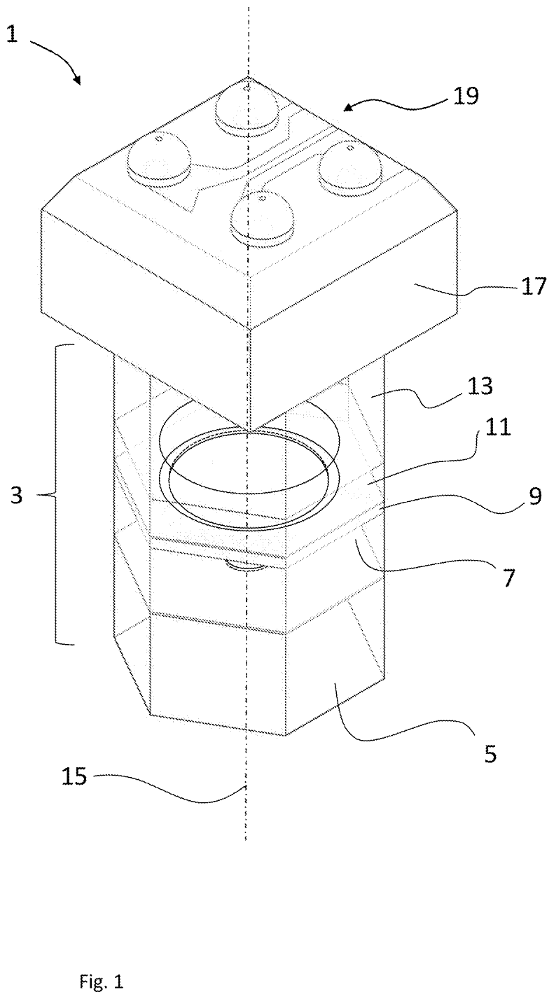

Referring to the drawings, FIG. 1 shows an objective lens 1 with an image sensor unit 17 arranged thereon for an endoscopic instrument. The objective lens 1 with an image sensor unit 17 arranged thereon, comprises an arrangement 3 of objective lens elements connected to each other separated out from a multilayer wafer package. The objective lens elements are preferably connected to each other in a materially bonded manner, e.g. adhered to each other. These are in the form of a first end plate 5, a glass substrate 7, a lens 9, a spacer 11 and a second end plate 13. The objective lens elements 5, 7, 9, 11 and 13 are stacked on top of each other along an optical axis 15. In this example of embodiment they each have a six-sided, equilateral, i.e. hexagonal cross-section perpendicular to the optical axis 15.

Also arranged on the second end plate 13 is an image sensor unit 17 which is designed to record an image produced by the lens 9 and to electrically provide it to signal connections 19 for processing and display. The lateral dimensions of the image sensor unit 17 are larger than the objective lens 1, but the optically effective area of the actual image sensor (not visible here) can be quadratic and a little smaller than the cross-section of the objective lens 1.

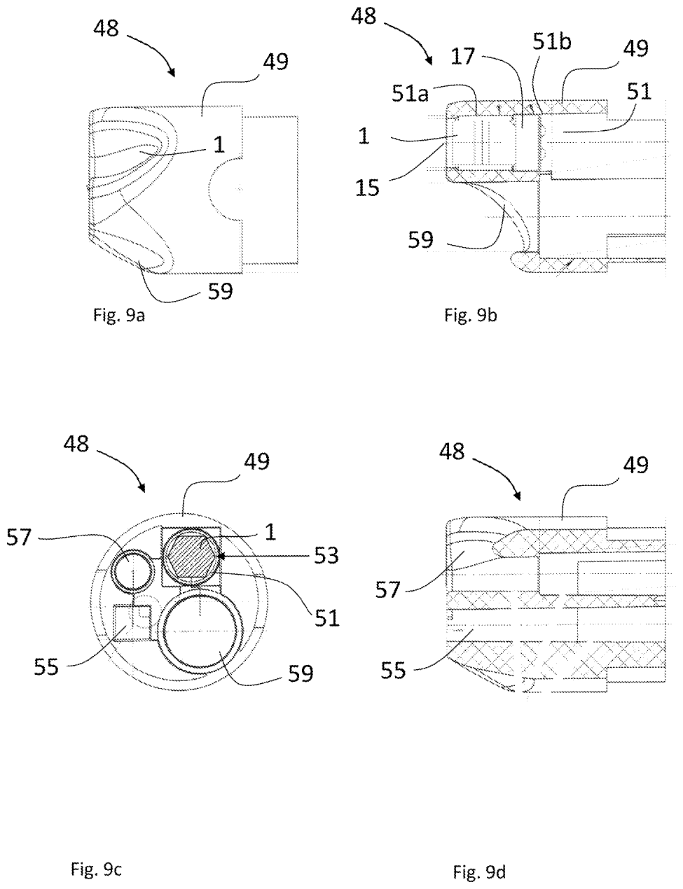

The advantage of the design according to the invention lies in the particularly good utilisation of the available cross-section of an imaging channel of an endoscopic instrument (see FIG. 9a-d), which usually has a circular cross-section. A polygonal cross-section which is at least hexagonal, can nestle against a circular contour comparatively well. Laborious post-processing to adapt it to an arrangement with a quadratic cross-section separated out from a wafer package is not necessary. Although two or more cuts are additionally needed for separating out of a wafer package, through the absence of post-processing the overall manufacturing costs of an objective lens can be considerably reduced compared with the prior art. A separate edging in addition to the imaging channel is also not necessary as such a polygonal contour can be easily integrated into an imaging channel in a friction-locking manner.

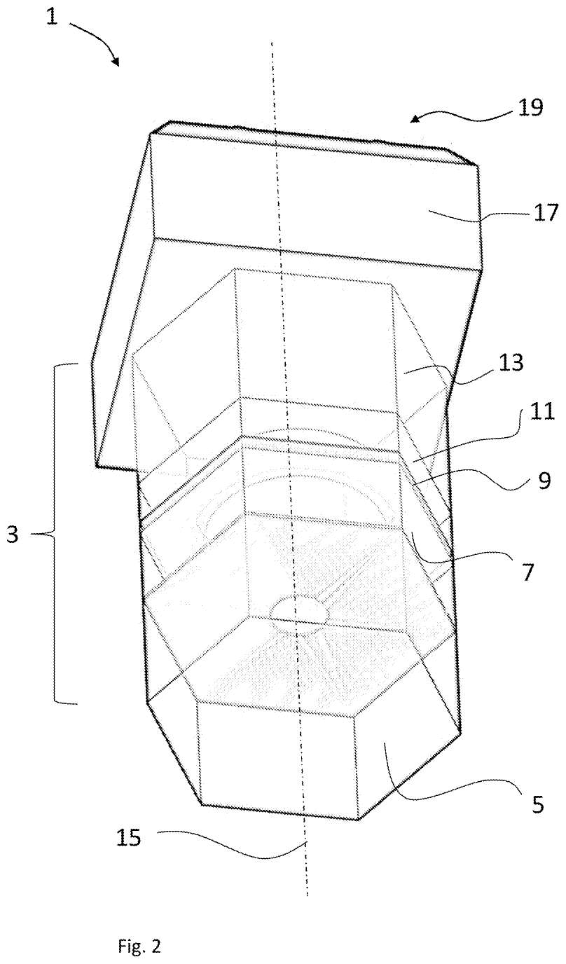

FIG. 2 shows the objective lens 1 from a perspective tilted in comparison with FIG. 1. On its underside facing the second end plate 13, the image sensor unit 17 is flat and lies flush on the second end plate 13.

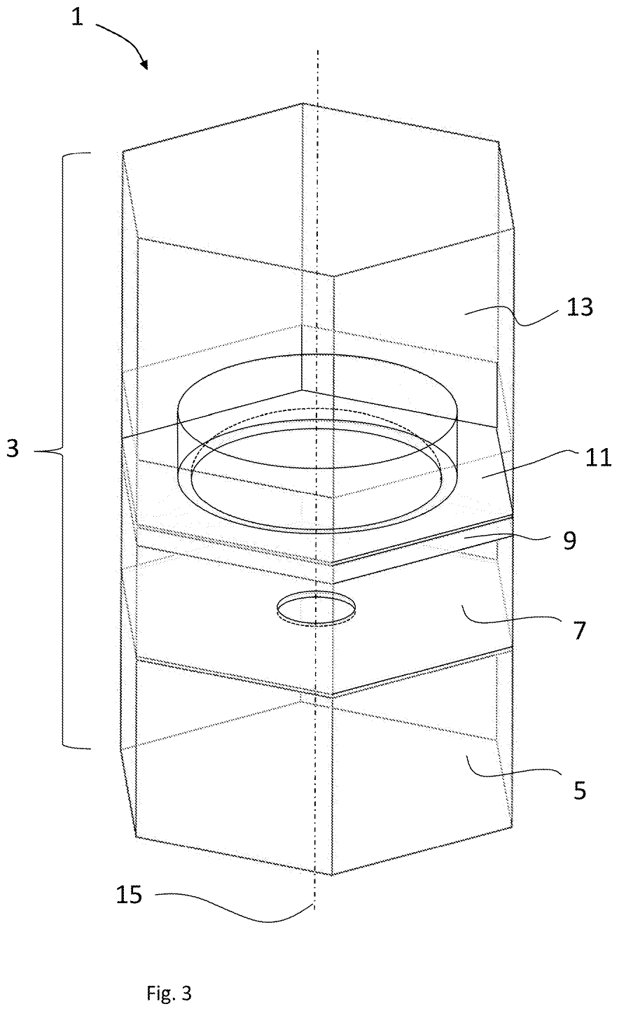

FIG. 3 shows the objective lens from the same perspective as in FIG. 1, but without the image sensor unit 17. Overall, the objective lens 1 has an elongated, cylindrical shape with a constant cross-section, wherein the objective lens elements 5, 7, 9, 11 and 13 have no circumferential edging.

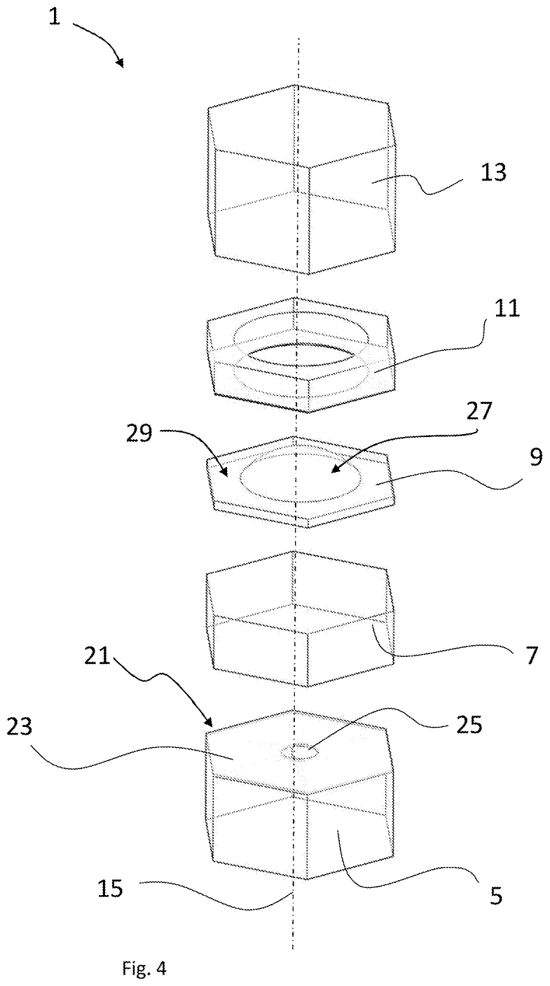

FIG. 4 shows the individual parts of the objective lens 1 in an exploded view. Here it can be seen that the first end plate 5 and the second endplate 13 each fill the cross-section over their height measured along the optical axis 15 completely and uninterruptedly. Arranged on a surface 21 of the first end plate 5 facing the glass substrate 7, or on a surface of the glass substrate 7 facing the end plate 5, there is a diaphragm in the form of a diaphragm coating 23 that is as matt as possible which covers the entire surface 21 apart from a, for example, circular diaphragm opening 25. The diaphragm coating 23 is not light permeable and could be made of chromium, chromium oxide, titanium, silicon, a polymer provided with dark particles or another material. The diaphragm coating 23 preferably has a light reflectance value of less than 10% upwards and/or downwards. The aperture opening 25 has a diameter adapted to the lens 9 and the image sensor unit 17.

The glass substrate 7 could be a carrier substrate for the lens 9 which can be built up from a UV-hardenable polymer on the glass substrate 7. The lens 9 has a preferably aspherically shaped surface 27 which gives the lens 9 a shape required for the desired light bundling. To protect the lens, the spacer 11 is arranged on a flat edge 29 around the aspherical surface 27 and extends along the optical axis 15 further than the aspherical surface 27. The second end plate 13 is provided at the end to cover the objective lens 1.

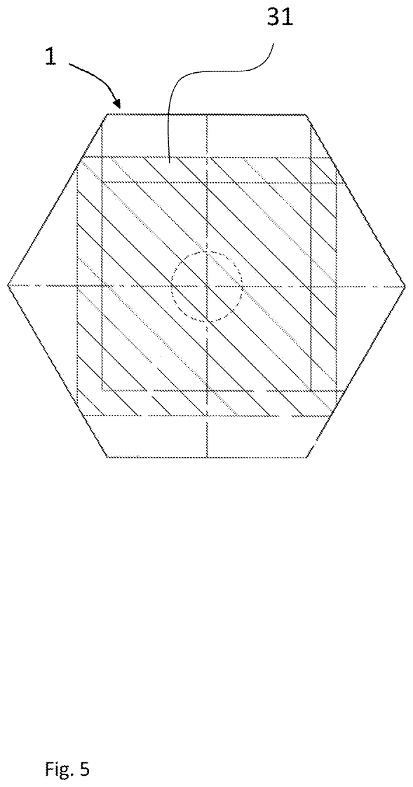

As an example, FIG. 5 shows a view from above of the objective lens 1 and an optically active surface 31 of an image sensor of the image sensor unit (17) (hatched). This surface 31 is quadratic and on the basis of the shape of the objective lens 1, which here is hexagonal as an example, can be selected to be sufficiently large. It is centred on the cross-section of the objective lens 1 and projects up to the lateral edges of the hexagonal cross-section arranged obliquely in the plane of the drawing.

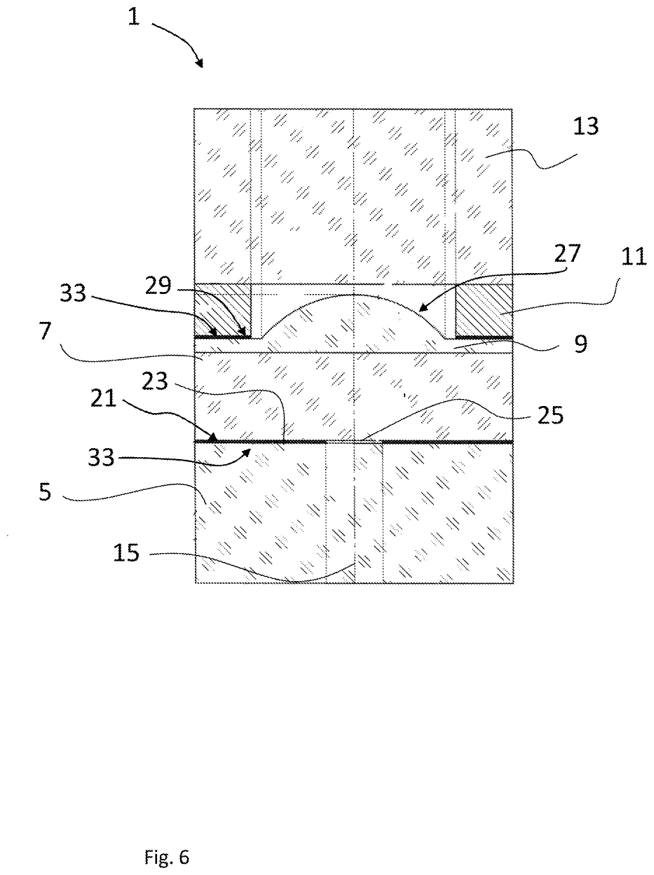

FIG. 6 shows a cross-sectional view of the objective lens 1. Provided between the first end plate 5 and the glass substrate 7 in addition to the diaphragm coating 23, there is also an adhesive layer 33 which connects the first end plate 5 to the glass substrate 7. The edge 29 is also connected to the spacer 11 in the same way. The spacer 11 and the second end plate 11 are, instead, connected to each other anodically, for example.

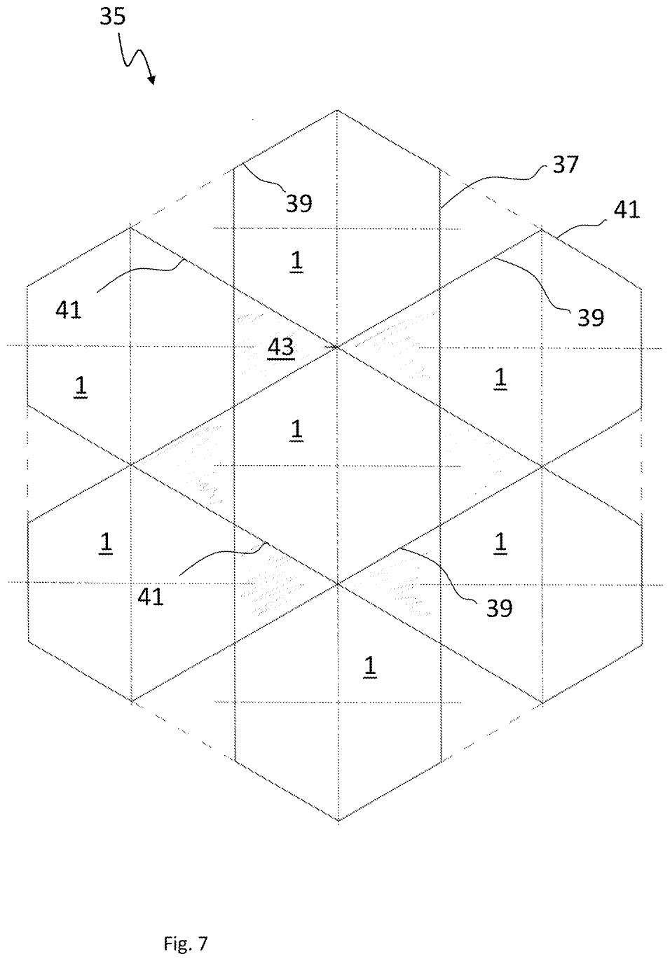

FIG. 7 show a section from a multilayer wafer package 35, wherein the individual layers each form a plurality of first plates 5, glass substrates 7, lenses 9, spacers 11 and second end plates 13. Here a series of separating cuts are shown, through which the individual objective lenses 1 can be separated out of the wafer package.

A group of first separating layers 37 runs in the vertical direction in the plane of the drawing. Several first separating cuts 37 are arranged in parallel to each other, wherein a distance between the midlines of the first separating cuts 37, corresponds to the distance between two opposites sides or surfaces of the objective lens 1. To form several hexagonal objective lenses 1, several second separating cuts 39 running in parallel to each other, and several third separating cuts 41 running in parallel to each other are provided. The distances of the second separating cuts 39 relative to each other and the distances of the third separating cuts 41 relative to each other are identical to the distance of the first separating cuts 37 relative to each other. The second separating cuts 39 and the third separating cuts 41 are each angled by an angle of 60° clockwise and anticlockwise respectively with regard to the first separating cuts 37.

Through alternating, column-wise offsetting in parallel to the first separating cuts 37 of the objective lens 1 to be separated out on the wafer package, very little in the way of offcuts is produced. Through this the hatched triangular offcut sections 43 arise. Over the used surface of the wafer package 35, for each objective lens 1 the total offcut areas corresponds to one third of the cross-sectional area of an objective lens 1. In differently-shaped polygons, this portion can deviate from this.

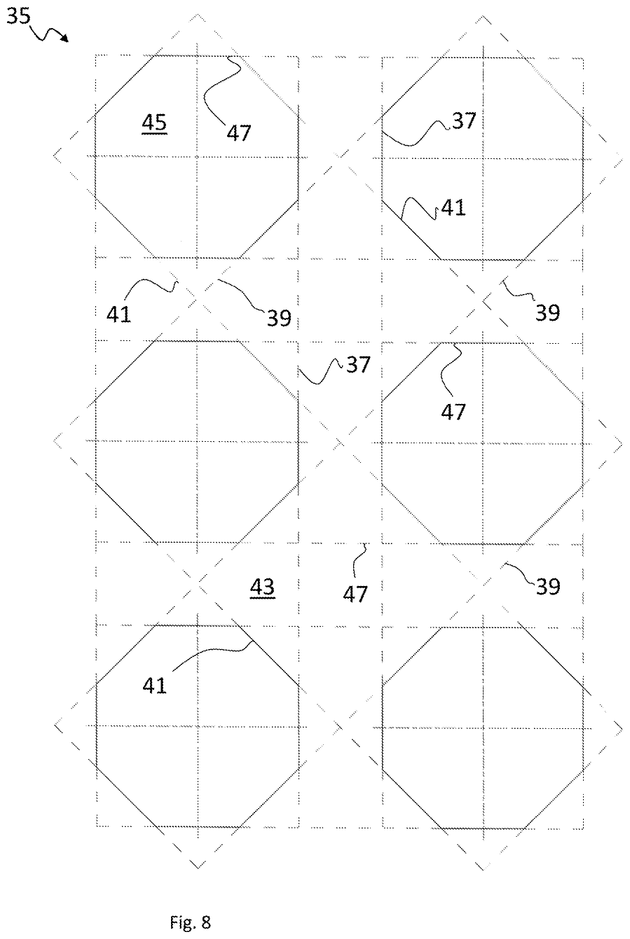

To produce octagonal objective lenses 45, FIG. 8 shows the possible separating cuts to be made. For this, in addition to first separating cuts 37, second separating cuts 39 and third separating cuts 41 as well as fourth separating cuts 47 must also be made. The second and third separating cuts are each angled by an angle of 45° clockwise and anticlockwise respectively with regard to the first separating cuts 37. The fourth separating cuts 47 are also arranged in parallel to each other and run vertically with regard to the first separating cuts 37.

FIGS. 9a to 9d show various views of a distal tip 49 of an endoscopic instrument 48. FIG. 9a is a side view, FIG. 9b a lateral section through the optical axis 15, FIG. 9c is view from above and FIG. 9d a lateral section through a working channel.

The distal tip 49 comprises an imaging channel 51, which on the distal end of the endoscopic instrument 48 has a circular cross-section. Here, the objective lens 1 from the preceding description is inserted in a friction-locking manner, so that the corners 53 of the cross-section of the objective lens 1 press flush into the imaging channel 51. The objective lens 1 can also be adhered in in order to seal off the imaging channel 51 outwards in a fluid-tight manner. The imaging channel 51 comprises a first distal imaging channel section 51a, into which the objective lens 1 is inserted, and, arranged proximally of the first imaging channel section 51a, is a second imaging channel section 51b which has a larger internal diameter than that the first imaging channel section 51a. The imaging sensor unit 17 materially bonded to the objective lens 1 is arranged in the second imaging channel section 51b. The lateral dimensions of the imaging sensor unit 17 arranged in the second imaging channel section 51b are slightly larger than the lateral dimensions of the objective lens 1, which is inserted to fit precisely into the first imaging channel section 51a. The objective lens 1 with the image sensor unit 17 is preferably fitted into the tip 49 as a preassembled unit from proximal, i.e. distally.

As can be seen in FIG. 9c, the distal tip 49 also has an illumination unit 55, which illuminates the area in front of the tip 49 in order to enable imaging thereof. For example, the illumination unit 55 can be in the form of an LED element with, for example, a rectangular or quadratic cross-section.

As well as the imaging channel 51 and the illumination unit 55, a first working channel 57 and a second working channel 59 end in the distal tip 49. The first working channel 57 has, for example, a considerably smaller cross-sectional area than the second working channel 59 and could, for example, be used for pushing through a laser light guide. The second working channel 59 can in the meantime be used for the as-required guiding of rinsing fluid and/or for an endoscopic tool.

FIG. 9b shows a section through the optical axis 15, i.e. the imaging channel 51, and shows the objective lens 1, on the proximal end of which the image sensor unit 17 is arranged. This is connected to an electrical lead, not shown here, which extends in the proximal direction. For the sake of completeness, in FIG. 9d a section through the first working channel 57 is shown.

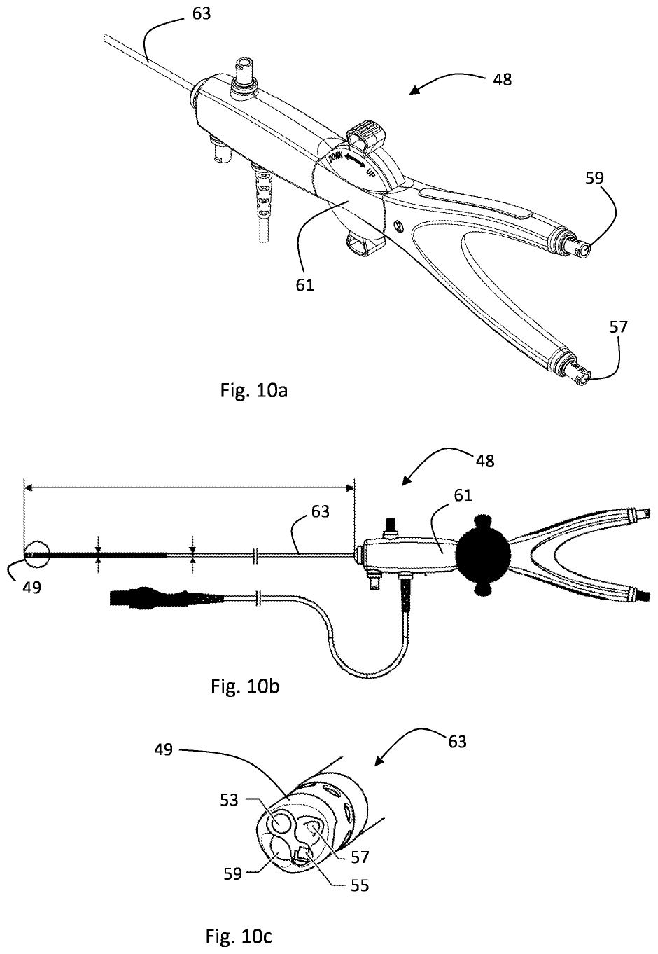

FIG. 10a shows a proximal part of the endoscopic instrument 48 with a manually useable handling device 61, from which a thin, rigid, tubular shaft 63 extends distally. The shaft 63 can preferably be angled or bent at a distal end section in order to be able to align the distal tip 49 of the shaft 63 as desired. In FIG. 10b the endoscopic instrument 48 is shown as a whole as a disposable article, wherein the shaft 63 is shown shortened. In reality, the shaft 63 is many times longer than the handling unit 61. FIG. 10c shows the distal tip 49 of the shaft of the endoscopic instrument 48, as shown in more detail in FIG. 9a-d.

While specific embodiments of the invention have been shown and described in detail to illustrate the application of the principles of the invention, it will be understood that the invention may be embodied otherwise without departing from such principles.

### LIST OF REFERENCE NUMBERS

1 Objective lens (hexagonal)
3 Arrangement
5 First end plate
7 Glass substrate
9 Lens
11 Spacer
13 Second end plate
15 Optical axis
17 Image sensor unit
19 Signal connections
21 Surface of the first end plate
23 Diaphragm coating/diaphragm
25 Diaphragm opening
27 Aspherical surface
29 Edge
31 Effective area
33 Adhesive layer
35 Wafer package
37 First separating layer
39 Second separating layer
41 Third separating layer
43 Offcut section
45 Lens (octagonal)
47 Fourth separating layer
48 Endoscopic instrument
49 Distal tip
51 Imaging channel
51a First imaging channel section
51b Second imaging channel section
53 corner
55 Illumination unit
57 First working channel
59 Second working channel
61 Handling device
63 Shaft

1. An endoscopic instrument, comprising:
a tubular shaft with a distal tip;
an objective lens at the distal tip of the tubular shaft, wherein the objective lens comprises an arrangement, separated out of a multilayer wafer package, of lens elements that are connected to each other and each have predetermined optical properties and follow each other along an optical axis, wherein the arrangement has a polygonal and at least hexagonal cross-section that is perpendicular to the optical axis, wherein the distal tip has an imaging channel, wherein the lens is inserted in the imaging channel in an interlocking, friction-locking and/or bonded manner, wherein the imaging channel comprises a first distal imaging channel section in which the objective lens is inserted, and a second imaging channel section arranged proximally of the first imaging channel section that has a greater inner diameter than the first imaging channel section; and
an image sensor unit connected in a bonded manner to the objective lens, wherein the image sensor unit is arranged in the second imaging channel section, wherein the image sensor unit has greater lateral dimensions than the objective lens.

2. An endoscopic instrument according to claim 1, wherein the cross-section of the objective lens is equilaterally polygonal and preferably hexagonal or octagonal.

3. An endoscopic instrument according to claim 1, wherein the objective lens elements comprise a first end plate and a second end plate which delimit the arrangement at end faces that are opposite to each other.

4. An endoscopic instrument according to claim 1, wherein the objective lens elements have comprise at least one lens.

5. An endoscopic instrument according to claim 4, wherein the lens has as an aspherically-shaped surface which is surrounded by a flat edge wherein a spacer is arranged on the edge and extends outwards along the optical axis over the aspherical surface.

6. An endoscopic instrument according to claim 4 or 5, wherein the lens is arranged on a flat and continuous surface of a glass substrate.

7. An endoscopic instrument according to claim 1, wherein the objective lens elements have at least one diaphragm.

8. An endoscopic instrument according to claim 7, wherein the diaphragm is arranged in front of the lens in the optical axis.

9. An endoscopic instrument according to claim 7 or 8, wherein the diaphragm has a front side and a rear side in relation to the optical axis, wherein the front side and/or the rear side has/have a light reflectance value of less than 10%.

10. An endoscopic instrument according to claim 1, wherein the image sensor unit is configured to record an optional image provided by the objective lens and make the optical image available in the form of electrical signals.

11. An endoscopic instrument according to claim 1, wherein at least a distal section of the shafts, that comprises the objective lens is configured as a disposable article.

12. An endoscopic instrument according to claim 1, wherein the objective lens is adhered in a fluid-tight manner to a distal opening of the imaging channel and thereby outwardly seals the imaging channel.

13. A method of manufacturing endoscopic instruments, the method comprising the steps:
providing several wafer layers of an optical material,
stacking of the layers to form a wafer package, and
separating out objective lenses from the wafer package by cutting the wafer package with at least three groups of parallel separating cuts, the incision directions of which are determined by a polygonal and at least hexagonal cross-section of all objective lenses,
providing a materially bonded connection of each of objective lenses with an image sensor unit, wherein the image sensor unit has greater lateral dimensions than the respective objective lens,
using each of the objective lenses materially bonded with the image sensor unit in an imaging channel of a distal tip of the endoscopic instruments to be manufactured, wherein the objective lens is distally inserted into the imaging channel in an interlocking, friction-locking and/or materially bonded manner, wherein the imaging channel comprises a first distal imaging channel section into which the objective lens is inserted, and, arranged proximally of the first imaging channel section, a second imaging channel section which for receiving the image sensor unit has a greater inner diameter than the first imaging channel section.

14. A method according to claim 13 wherein when separating out the objective lenses, at least three groups of parallel separating cuts are made, wherein a first incision direction and a second incision direction as well as the second incision direction and a third incision direction each enclose an angle of 60°.
